(19) United States
(12) Patent Application Publication
(10) Pub. No.: US 2016/0271426 A1
(43) Pub. Date: Sep. 22, 2016

(54) Ultrasound on stomach blockages

(71) Applicant: Michael Lewis Moravitz, Fairfax, VA (US)

(72) Inventor: Michael Lewis Moravitz, Fairfax, VA (US)

(21) Appl. No.: 14/999,537

(22) Filed: May 23, 2016

(51) Int. Cl. A61N 7/00

(52) U.S. Cl. CPC A61N 7/00; A61N 2007/0004

(57) ABSTRACT

Ultrasound technology, which is used to break apart kidney stones, might also be used to break up stomach blockages rather than a colonoscopy in some cases.

Ultrasound devices, which can be used to break apart kidney stones in a patient, might also be used to break down stomach blockages for purging.

Research is necessary into this potentially less invasive and more cost effective procedure than a colonoscopy or other intestinal surgery. This treatment might be best for large blockages, but the strength of the sound waves necessary to undue such maladies needs to be determined. This also might be a preferred treatment than having a microscopic device sent up one's colon. If this patent is approved, I deserve the Nobel Prize in Medicine. So I hope everything is processed correctly and comes out well.

1) Patents for ultrasound devices.
